(19) United States
(12) Patent Application Publication
(10) Pub. No.: US 2009/0120824 A1
(43) Pub. Date: May 14, 2009

(54) DIAPER PACKAGE WITH PLURALITY OF INDIVIDUAL DIAPER PACKAGES VACUUM PACKED THEREIN

(76) Inventor: John Piazza, Winston Salem, NC (US)

Correspondence Address: TILLMAN WRIGHT, PLLC, PO BOX 473909, CHARLOTTE, NC, 28247, US

(21) Appl. No.: 12/233,306

(22) Filed: Sep. 18, 2008

Related U.S. Application Data
(60) Provisional application No. 60/973,307, filed on Sep. 18, 2007.

(51) Int. Cl. A61B 19/02; B65B 31/00

(52) U.S. Cl. 206/438; 53/434; 53/432

(57) ABSTRACT

The invention relates to diaper packaging and, more particularly, to vacuum packing together a plurality of individual diaper packages. The invention also relates to an overall package containing a plurality of individual diaper packages, wherein each individual diaper package is a vacuum packed diaper (VPD). In accordance therewith, a method of packaging diapers includes the steps of: (a) individually packing each of a plurality of diapers to form a plurality of individual diaper packages; and thereafter (b) vacuum packing the plurality of individual diaper packages together to form an overall package of diapers. In a feature, each individual diaper package includes only a single vacuum packed diaper. In another feature, a retail package for sale to consumers includes a group of non vacuum packed diapers, none of which are individually packaged, and an overall package of VPDs.

### CROSS-REFERENCE TO RELATED APPLICATION

The present application is a U.S. nonprovisional patent application of, and claims priority under 35 U.S.C. § 119(e) to, U.S. provisional patent application Ser. No. 60/973,307, filed Sep. 18, 2007, which provisional patent application is hereby incorporated herein by reference.

### COPYRIGHT STATEMENT

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

### BACKGROUND OF THE INVENTION

Individually vacuum packing a diaper for consumer convenience in transport and storage and, in particular, vacuum packing a diaper for carrying in a pocket on one's person is disclosed, for example, in Corlett U.S. Patent Application Publication No. US 2006-0206078 A1, which is incorporated herein by reference.

Furthermore, a diaper kit having both a vacuum packed diaper (VPD) and accessories for a diaper changing, which diaper kit is pocketable for carrying in a pocket on one's person, is disclosed for example in Snell U.S. Patent Application Publication No. US 2004-0176735 A1, which is incorporated herein by reference.

These patent references represent the recent recognition of the advantages and benefits that are provided by VPDs to consumers.

The need to facilitate consumer recognition and acceptance of VPDs also has been identified, for example, in Snell U.S. Pat. Nos. 7,194,848 B2 and 7,181,894 B2, each of which is incorporated herein by reference. In particular, the invention claimed in the Snell '848 patent serves to reduce wrinkles in a surface of the encasement in which a diaper has been vacuum packed. The wrinkles are reduced in the surface of the encasement, inter alia, by pressing the encasement after vacuum packing the diaper. Reducing wrinkles enhances the consumer appearance of the VPD.

The invention claimed in the Snell '894 patent, among other things, also tends to reduce wrinkles. In particular, the claimed method includes foldingly reducing the dimensions of a diaper and then compressing the height of the folded diaper in order to reduce its height or thickness. The volumetrically reduced diaper is then vacuum packed within an encasement, and the encasement includes a volume and dimensions that closely match the volume and dimensions of the foldingly reduced diaper. Because the volume and dimensions of the encasement closely match the volume and dimensions of the foldingly reduced diaper, it is believed that excess material of the encasement following vacuum packing is reduced and, consequently, that the quantity and extent of wrinkles formed in the material of the encasement following vacuum packing is reduced.

While Corlett and Snell represent significant advances in the field of diaper packaging, a need nevertheless exists for continued improvement in VPDs and the commercialization thereof in consumer markets. This, and other needs, are addressed by one or more aspects of the present invention.

### FIELD OF THE INVENTION

The field of the invention is that of diaper packaging.

### SUMMARY OF THE INVENTION

The invention relates to diaper packaging and, more particularly, to vacuum packing a plurality of individual diaper packages. The invention also generally relates to a package containing a plurality of individually vacuum packed diapers which package itself is vacuum packed.

The invention includes many aspects and features.

One aspect of the invention relates to a method of packaging diapers that includes the steps of: (a) individually packing each of a plurality of diapers to form a plurality of individual diaper packages; and thereafter (b) vacuum packing together the plurality of individual diaper packages in order to form an overall package of diapers.

In a feature of this aspect, the step of vacuum packing together the plurality of individual diaper packages includes vacuum sealing the plurality of individual diaper packages within a substantially air impermeable material such that the individual packaged diapers are all contained within a substantially airtight interior space of the overall package.

In another feature of this aspect, the step of individually packing each of the plurality of diapers includes vacuum sealing each of the plurality of diapers within a substantially air impermeable material such that the only diaper vacuum packed therein is contained within a substantially airtight interior space of the individual diaper package.

In another feature of this aspect, each of the individual packaged diapers is contained within its respective package with the interior space thereof having a first pressure, and the plurality of individual diaper packages are contained within the overall package with the interior space thereof having a second pressure. The first pressure and the second pressure are each less than ambient atmospheric pressure. Furthermore, in variations of this aspect, the first pressure is greater than the second pressure; the first vacuum is substantially the same the second pressure; and the first pressure is less than the second pressure.

In another aspect of the invention, a method of packaging diapers includes the steps of: (a) individually vacuum packing each of a plurality of diapers within a substantially air impermeable material to form a plurality of individually vacuum packed diapers (VPDs); and thereafter (b) vacuum packing the plurality of VPDs together within a substantially air impermeable material.

In a feature of this aspect, each of the diapers is vacuum packed at a first vacuum pressure, and the plurality of VPDs are vacuum packed together at a second vacuum pressure. In variations of this aspect, the first vacuum pressure is greater than the second vacuum pressure; the first vacuum pressure is substantially the same the second vacuum pressure; and the first vacuum pressure is less than the second vacuum pressure.

In a feature of this aspect, each VPD includes an encasement that is formed prior to vacuum sealing of a single diaper therein, and the diaper is compressed prior to being located within the encasement for vacuum sealing. Additionally, the encasement includes a volume and dimensions that closely match the volume and dimensions of the compressed diaper when located within the encasement such that the encasement retains the diaper substantially in the compressed state prior to vacuum sealing of the diaper within the encasement.

In a feature of this aspect of the invention, each encasement within which a respective diaper is individually vacuum sealed includes a hoop strength that is sufficient to retain the diaper therein and prevent breach of the vacuum therein when the VPD is subsequently exposed to extremely low pressure during vacuum packing thereof with other VPDs to form the overall package of VPDs.

In another feature of this aspect, the vacuum sealing of a diaper is performed using a vacuum chamber.

In another feature of this aspect, the vacuum sealing of the plurality of VPDs is performed using a vacuum chamber.

In another feature of this aspect, during the process of vacuum packing the plurality of VPDs together, the plurality of VPDs are pressed when the VPDs are exposed to vacuum. Furthermore, the plurality of VPDs may be pressed while sealing of the VPDs within the substantially air impermeable material. The plurality of VPDs also may be pressed while initially being exposed to atmospheric pressure after vacuum sealing. Any such pressing may be accomplished with one or more substantially smooth surfaces.

In still yet another feature of this aspect, the package of VPDs is pressed following vacuum packing. The pressing may be accomplished with a substantially smooth surface.

In yet another feature, each diaper is arranged in the nominal configuration when vacuum sealed within a respective encasement. The diaper in the nominal configuration also first may be compressed before vacuum sealing. A VPD in which the diaper has been vacuum packed in the nominal configuration (whether compressed prior to vacuum packing) is sometimes referred to as a “flat pack” because of the remarkable thinness of the resulting package.

In another feature of this aspect, each of a plurality of VPDs comprises a flat pack, and the VPDs are vacuum packed together to form an overall package that is generally rectangular or “brick” shaped. In this regard, each flat pack represents a “layer” of the brick. Such a package is sometimes referred to herein as a “diaper brick” and preferably includes five flat packs.

In another aspect of the invention, an overall diaper package includes a plurality of VPDs collectively vacuum packed together in accordance with the foregoing aspect and features.

In another aspect of the invention, an overall package of a plurality of individual vacuum packed diapers comprises a subset of the total number of diapers within a retail package for sale to a consumer. In this regard, the retail package of diapers includes the combination of individually vacuum packed diapers and other packed diapers. Such combinations may be useful as disclosed, for example, in each of Snell U.S. patent application Ser. No. 10/907,389 (and the application publication thereof and Snell International Patent Application Publication No. WO 2006/099371 A1, both of which are hereby incorporated herein by reference.

In another feature of the foregoing aspects, each individual diaper package includes a dense and hard exterior surface resulting from vacuum packing of the diaper thereof and/or the overall package of the individual diaper packages includes a hard exterior surface following the vacuum packing thereof.

In addition to these aspects and features and those disclosed below and in the drawings, it should be understood that the invention further encompasses the various possible combinations of such aspects and features.

### BRIEF DESCRIPTION OF THE DRAWINGS

One or more preferred embodiments of the invention now will be described in detail with reference to the accompanying drawings, wherein the same elements are referred to with the same reference numerals, and wherein:

FIG. 1A illustrates steps of a method in accordance with a first embodiment of the invention.

FIG. 1B illustrates steps of a method in accordance with a second embodiment of the invention.

FIG. 2A is a side plan view of an individual diaper package comprising a vacuum packed diaper in accordance with an embodiment of the invention.

FIG. 2B is a side plan view of the individual diaper package of FIG. 2A, wherein the encasement of the individual diaper package is partially omitted in order to show the volumetrically reduced diaper contained therein.

FIG. 3A is a side plan view of an overall package in which a plurality of individual diaper packages are contained in accordance with an embodiment of the invention.

FIG. 3B is a side plan view of the overall package of FIG. 3A, wherein an encasement of the overall package is partially omitted in order to show the individual diaper packages contained therein.

FIG. 4A is a side plan view of a retail package of diapers for sale to a consumer in accordance with an embodiment of the invention, wherein the diapers include both non vacuum packed diapers and two overall packages each containing a plurality of individual diaper packages vacuum packed together in accordance with an embodiment of the invention.

FIG. 4B is a side plan view of the retail package of FIG. 4A, wherein an encasement of the retail package is partially omitted in order to show the non vacuum packed diapers and the two packages of VPDs contained therein.

FIG. 4C is a side plan view of the retail package of FIG. 4A, wherein both the encasement of the retail package and an encasement of each of the overall packages of VPDs are partially omitted in order to show the non vacuum packed diapers and the vacuum packed diapers of the retail package of FIG. 4A.

FIG. 5 is a perspective view of an exemplary disposable diaper arranged in its nominal configuration.

FIG. 6 is a top plan view of vacuum packed diaper containing the diaper of FIG. 5.

FIG. 7 is a side perspective view of the vacuum packed diaper of FIG. 6.

### DETAILED DESCRIPTION

As a preliminary matter, it will readily be understood by one having ordinary skill in the relevant art (“Ordinary Artisan”) that the invention has broad utility and application. Furthermore, any embodiment discussed and identified as being “preferred” is considered to be part of a best mode contemplated for carrying out the invention. Other embodiments also may be discussed for additional illustrative purposes in providing a full and enabling disclosure of the invention. Moreover, many embodiments, such as adaptations, variations, modifications, and equivalent arrangements, will be implicitly disclosed by the embodiments described herein and fall within the scope of the invention.

Accordingly, while the invention is described herein in detail in relation to one or more embodiments, it is to be understood that this disclosure is illustrative and exemplary of the invention, and is made merely for the purposes of providing a full and enabling disclosure of the invention. The detailed disclosure herein of one or more embodiments is not intended, nor is to be construed, to limit the scope of patent protection afforded the invention, which scope is to be defined by the claims and the equivalents thereof. It is not intended that the scope of patent protection afforded the invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the invention. Accordingly, it is intended that the scope of patent protection afforded the invention is to be defined by the appended claims rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

Furthermore, it is important to note that, as used herein, “a” and “an” each generally denotes “at least one,” but does not exclude a plurality unless the contextual use dictates otherwise. Thus, reference to “a picnic basket having an apple” describes “a picnic basket having at least one apple” as well as “a picnic basket having apples.” In contrast, reference to “a picnic basket having a single apple” describes “a picnic basket having only one apple.”

When used herein to join a list of items, “or” denotes “at least one of the items,” but does not exclude a plurality of items of the list. Thus, reference to “a picnic basket having cheese or crackers” describes “a picnic basket having cheese without crackers”, “a picnic basket having crackers without cheese”, and “a picnic basket having both cheese and crackers.” Finally, when used herein to join a list of items, “and” denotes “all of the items of the list.” Thus, reference to “a picnic basket having cheese and crackers” describes “a picnic basket having cheese, wherein the picnic basket further has crackers,” as well as describes “a picnic basket having crackers, wherein the picnic basket further has cheese.”

Furthermore, as used herein—and without regard to anything in the incorporated references to the contrary—the word “diaper” means any one of a disposable diaper, an adult incontinent disposable diaper, training pants, and pull-ups, and any equivalent thereof.

Similarly, the “nominal configuration” is defined to be the bifolded configuration of the diaper with side margins (which typically include elastic nonabsorbent portions) tucked within the sides thereof. This configuration is well known, as diapers are conventionally packaged in this configuration for retail sale to consumers. Examples of the bifolded configuration may be readily seen, for example, from the disclosure of Snell International Patent Application Publication No. WO 2006/099370 A1, which is hereby incorporated herein by reference; and Kellenberger US 2004-0167489 A1, which is hereby incorporated herein by reference.

Referring now to the drawings, one or more preferred embodiments of the invention are next described. The following description of one or more preferred embodiments is merely exemplary in nature and is in no way intended to limit the invention, its implementations, or uses.

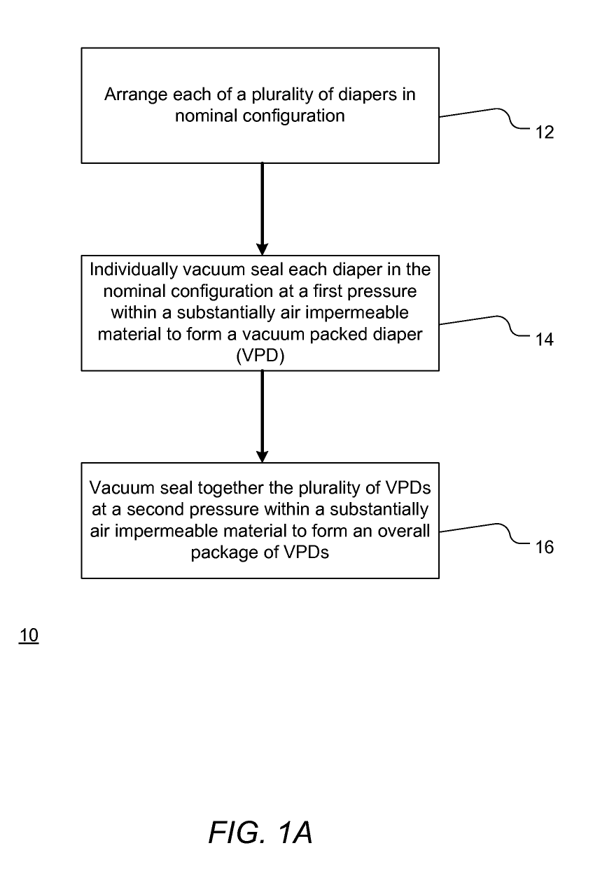

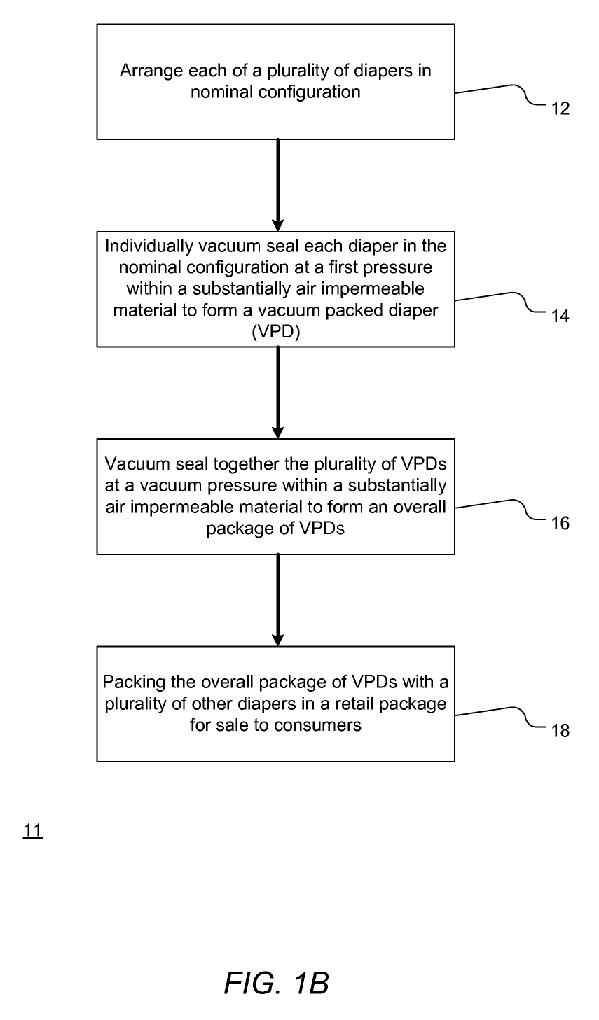

With specific reference to FIG. 1A, steps of a method 10 of packaging a plurality of individual vacuum packed diapers (VPDs) in accordance with a preferred embodiment of the invention are illustrated.

In step 12 each of a plurality of diapers is arranged in a nominal configuration.

In step 14, each diaper arranged in the nominal configuration is vacuum sealed within a substantially air impermeable material to form a VPD. The vacuum sealing preferably is accomplished using a vacuum sealing machine having a vacuum chamber. The resulting VPD includes a substantially air impermeable encasement defining a substantially airtight interior space in which the diaper is located, and the interior space has a first pressure that is less than ambient atmospheric pressure.

In step 16, each of a plurality of VPDs formed in accordance with step 14 are vacuum packed together. In this regard, the plurality of VPDs are vacuum sealed within a substantially air impermeable material to form an overall package of VPDs. The resulting overall package includes a substantially air impermeable encasement defining a substantially airtight interior space in which the VPDs are located, and the interior space has a second pressure that is less than ambient atmospheric pressure. In variations, the second pressure may be less than, equal to, or greater than the first pressure in accordance with variations of the invention.

It is believed that one or more of the foregoing variations may result in advantages in packaging and presentation of the VPDs to a consumer and/or advantages in shipping, transport, and storage of the VPDs.

FIG. 1B illustrates steps of a method 11 of packaging a plurality of VPDs in accordance with another preferred embodiment of the invention. Method 11 includes as a subcombination the steps 12, 14, 16 of method 10.

In addition to these steps, method 11 further includes step 18, in which an overall package of VPDs formed in accordance with steps 12, 14, 16 is packed with a plurality of other diapers in a retail package for sale to consumers. Preferably, the other diapers are not individually packaged and are not individually vacuum packed. The other diapers preferably are simply contained within an interior space of the film wrapping of the retail package.

On the other hand, it is contemplated that the overall package of VPDs and the plurality of other diapers may, in fact, be vacuum sealed together in a substantially air impermeable material in yet a third vacuum packing process. It is believed that such third vacuum packing process may result in yet additional advantages in packaging and presentation of the VPDs to a consumer and/or advantages in shipping, transport, and storage of the retail package.

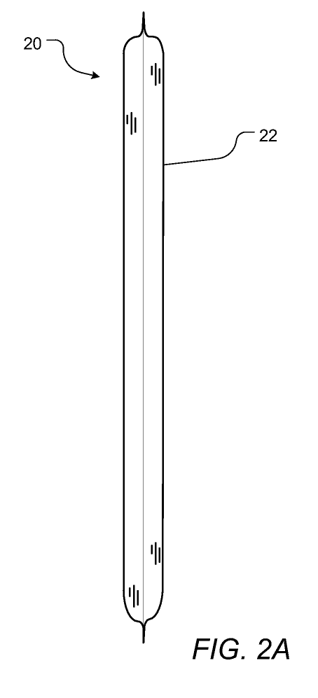

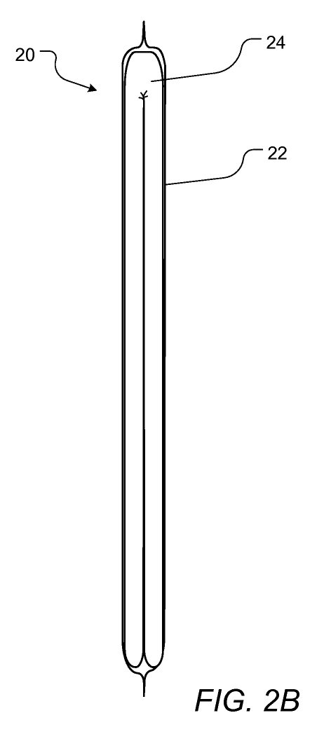

With regard to the individual diaper packages, an exemplary such package is illustrated in FIGS. 2A-2B. In this regard, FIG. 2A is a side plan view of an individual diaper package 20 comprising a diaper 24 vacuum packed in accordance with an embodiment of the invention. FIG. 2B is a side plan view of the individual diaper package 20 of FIG. 2A, wherein a substantially air impermeable encasement 22 of the individual diaper package 20 is partially omitted in order to show the diaper 24 contained therein.

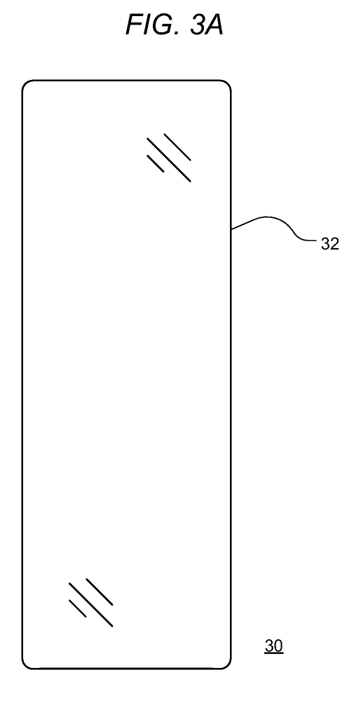

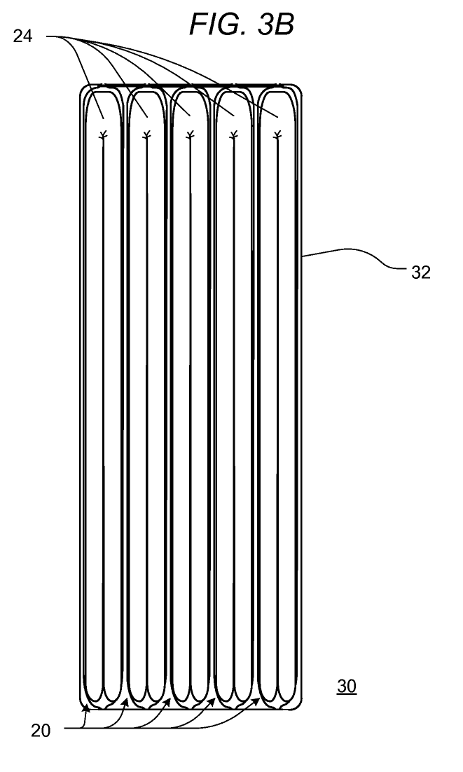

FIG. 3A is a side plan view of an overall package 30 containing a plurality of individual diaper packages 20 in accordance with an embodiment of the invention. FIG. 3B is a side plan view of the overall package 30 of FIG. 3A, wherein an encasement 32 of the overall package 30 is partially omitted in order to show the individual diaper packages 20 contained therein, each diaper package 20 including a diaper 24.

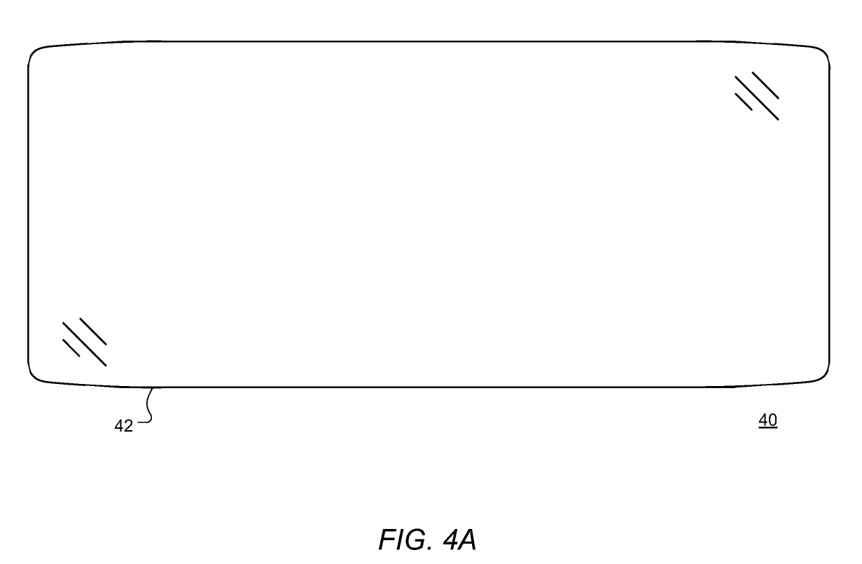

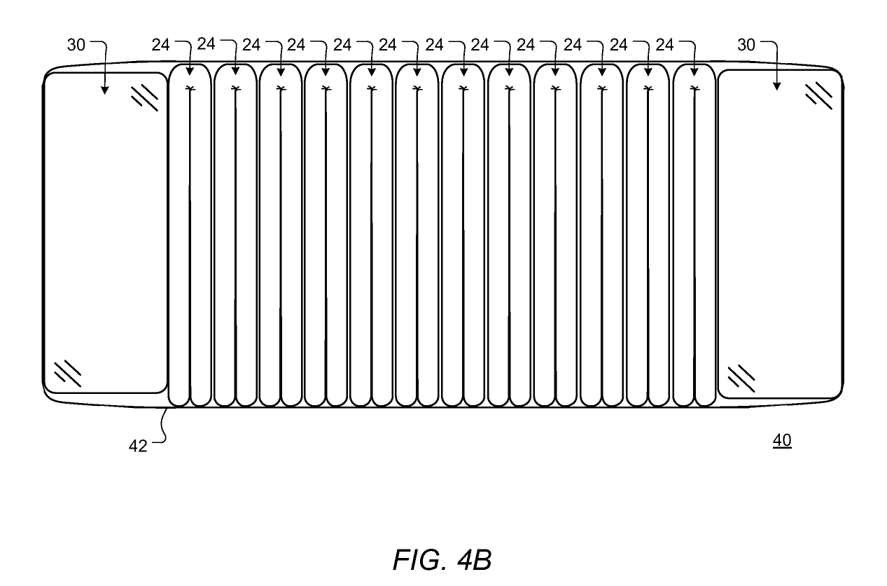

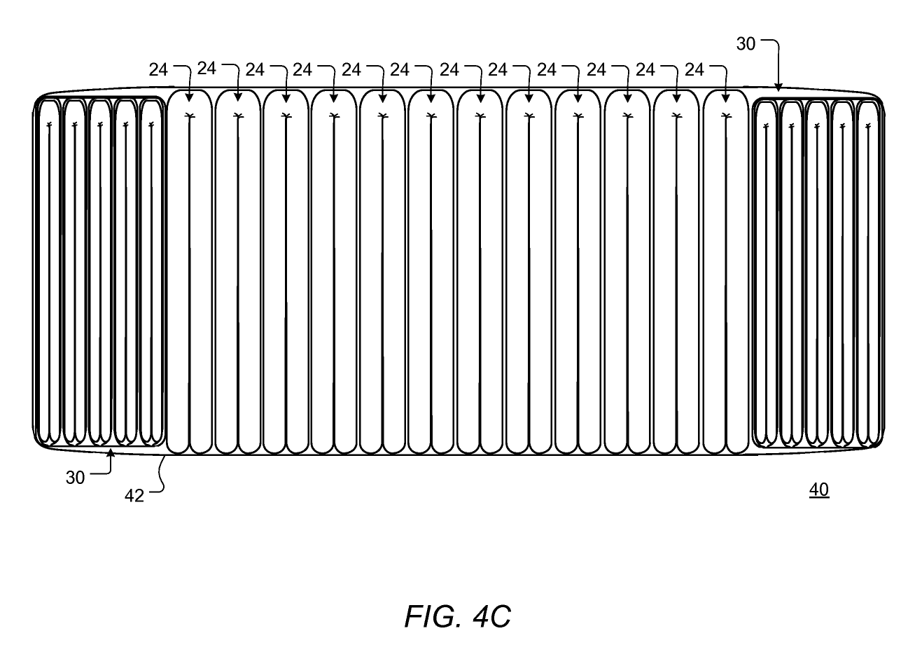

FIG. 4A is a side plan view of a retail package 40 of diapers for sale to a consumer in accordance with an embodiment of the invention, wherein the diapers 24 in the retail package 40 include both non vacuum packed diapers and two packages 30 each containing a plurality of individual diaper packages 20 in accordance with an embodiment of the invention. FIG. 4B is a side plan view of the retail package 40 of FIG. 4A, wherein an encasement 42 of the retail package 40 is partially omitted in order to show the diapers 24 that are not vacuum packed and the two packages 30 each containing a plurality of individual diaper packages 20. The diapers 24 of the individual diaper packages 30 are further illustrated in FIG. 4C.

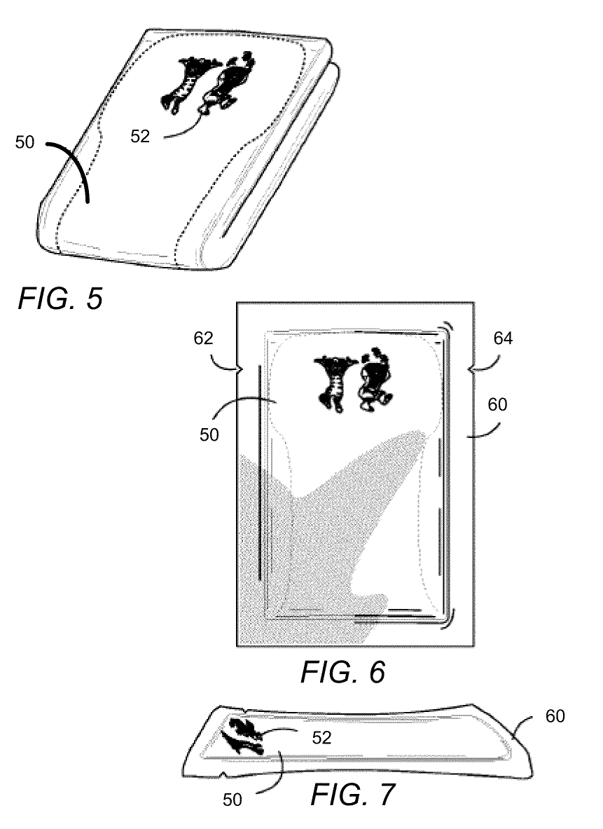

For purposes of clarity of description with regard to the “nominal configuration” of a disposable diaper, FIG. 5 is a perspective view of an exemplary disposable diaper 50 arranged in the nominal configuration; FIG. 6 is a top plan view of the diaper 50 after vacuum packing; and FIG. 7 is a side perspective view of the diaper 50 after vacuum packing.

The diaper 50 includes a crosswise fold located approximately at the middle of the diaper 50 such that top and bottom overlapping portions of the diaper 50 are substantially coextensive. The diaper 50 is vacuum sealed within a flexible, substantially air impermeable encasement 60, and the resulting vacuum packed diaper is remarkably thin.

Furthermore, as shown, the encasement 60 is transparent such that graphics 52 printed on the diaper 50 are visible through the encasement 60 following vacuum packing. Notches 62, 64 formed in the encasement 60 are also shown, which notches 62, 64 facilitate opening and removal of the diaper 50 from the encasement 60.

Based on the foregoing description, it will be readily understood by those persons skilled in the art that the invention is susceptible of broad utility and application. Many embodiments and adaptations of the invention other than those specifically described herein, as well as many variations, modifications, and equivalent arrangements, will be apparent from or reasonably suggested by the invention and the foregoing descriptions thereof, without departing from the substance or scope of the invention. Accordingly, while the invention has been described herein in detail in relation to one or more preferred embodiments, it is to be understood that this disclosure is only illustrative and exemplary of the invention and is made merely for the purpose of providing a full and enabling disclosure of the invention. The foregoing disclosure is not intended to be construed to limit the invention or otherwise exclude any such other embodiments, adaptations, variations, modifications or equivalent arrangements, the invention being limited only by the claims appended hereto and the equivalents thereof.

What is claimed is:

1. A method of packaging diapers, comprising the steps of:
(a) individually packing each of a plurality of diapers to form a plurality of individual diaper packages; and
(b) thereafter, vacuum sealing together the plurality of individual diaper packages within a substantially air impermeable material to form an overall package of diapers.

2. A method of packaging diapers, comprising the steps of:
(a) individually vacuum sealing each of a plurality of diapers within a substantially air impermeable material such that each respective diaper is contained within a substantially airtight interior space of a respective diaper package so as to form a plurality of vacuum packed diapers (VPDs); and
(b) thereafter, vacuum sealing together the plurality of VPDs within a substantially air impermeable material such that the plurality of VPDs are contained within a substantially airtight interior space of the overall package.

3. The method of claim 2, wherein each of the diapers is vacuum sealed in said step (a) at a first vacuum pressure, wherein the plurality of VPDs are vacuum sealed in said step (b) at a second vacuum pressure, and wherein the first vacuum pressure is substantially greater than the second vacuum pressure.

4. The method of claim 3, wherein each of the diapers is vacuum sealed in said step (a) at a first vacuum pressure, wherein the plurality of VPDs are vacuum sealed at a second vacuum pressure, and wherein the first vacuum pressure is substantially less than the second vacuum pressure.

5. The method of claim 3, wherein each of the diapers is vacuum sealed in said step (a) at a first vacuum pressure, wherein the plurality of VPDs are vacuum sealed at a second vacuum pressure, and wherein the first vacuum pressure is substantially the same as the second vacuum pressure.

6. The method of claim 3, wherein each of the diapers is vacuum sealed in said step (a) at a first pressure, wherein the plurality of VPDs are vacuum sealed at a second pressure, and wherein the first pressure is less than ambient atmospheric pressure and wherein the second pressure is less than ambient atmospheric pressure.

7. The method of claim 3, wherein each VPD comprises an encasement in which the diaper thereof is vacuum sealed, the encasement is formed from the substantially air impermeable material prior to vacuum sealing of the respective diaper therein, and the diaper is compressed prior to being located within the encasement for vacuum sealing.

8. The method of claim 7, wherein the encasement includes a volume and dimensions that closely match the volume and dimensions of the compressed diaper when located within the encasement such that the encasement retains the diaper substantially in the compressed state prior to vacuum sealing of the diaper within the encasement.

9. The method of claim 3, wherein each VPD comprises an encasement in which the diaper thereof is vacuum sealed, the encasement is formed from the substantially air impermeable material prior to vacuum sealing of the respective diaper, and each encasement in which a diaper is vacuum sealed includes a hoop strength that is sufficient to retain the diaper therein and prevent breach of a vacuum state therein when the encasement is exposed to extremely low pressure during vacuum packing thereof in said step (b) to form the overall package of VPDs.

10. The method of claim 3, wherein at least one of said steps (a) and (b) is performed using a vacuum chamber.

11. The method of claim 3, further comprising the step of pressing the plurality of VPDs during performance of said step (b).

12. The method of claim 11, wherein said further step of pressing the plurality of VPDs comprises pressing VPDs with one or more substantially smooth surfaces.

13. The method of claim 3, further comprising the step of pressing the plurality of VPDs when the VPDs are exposed to vacuum during performance of said step (b).

14. The method of claim 3, further comprising pressing the plurality of VPDs while sealing of the VPDs within the substantially air impermeable material during performance of said step (b).

15. The method of claim 3, further comprising pressing the plurality of VPDs while initially exposing the overall packaging to ambient atmospheric pressure during performance of said step (b).

16. The method of claim 3, further comprising pressing the plurality of VPDs after performance of said steps (a) and (b).

17. The method of claim 3, wherein each diaper is arranged in a nominal configuration when individually vacuum packed during performance of said step (a).

18. The method of claim 3, further comprising compressing a diaper that is arranged in a nominal configuration prior to vacuum sealing of the diaper during performance of said step (a), wherein the diaper is in a compressed state when vacuum sealed within the substantially air impermeable material during performance of said step (a).

19. The method of claim 3, wherein the overall package resulting from the performance of said step (b) consists of five VPDs.

20. The method of claim 3, wherein following performance of said step (b), the overall package of VPDs has a hard exterior surface that results from the vacuum sealing of the VPDs within the substantially air impermeable material.

21. The method of claim 3, wherein following performance of said step (a), each of the VPDs has a hard exterior surface that results from the individual vacuum sealing of each diaper of the VPDs.

22. A retail package of diapers for sale to a consumer comprising a plurality of non vacuum packed diapers and one or more overall packages, each respective overall package comprising individual vacuum packed diapers (VPDs), wherein the VPDs are collectively vacuum sealed together within a substantially air impermeable encasement to form the respective overall package.
